(19) United States
(12) Patent Application Publication
(10) Pub. No.: US 2025/0228681 A1
(43) Pub. Date: Jul. 17, 2025

(54) STENT DELIVERY SYSTEM HAVING STENT CONSTRAINING STRUCTURE

(71) Applicant: BOSTON SCIENTIFIC SCIMED, INC., Maple Grove, MN (US)

(72) Inventors: MARTYN G. FOLAN, Galway (IE); LOUIS MCNERN, Donegal (IE); KEVIN MCEVILLY, Galway (IE)

(73) Assignee: BOSTON SCIENTIFIC SCIMED, INC., Maple Grove, MN (US)

(21) Appl. No.: 19/022,345

(22) Filed: Jan. 15, 2025

Related U.S. Application Data
(60) Provisional application No. 63/621,400, filed on Jan. 16, 2024.

(51) Int. Cl. A61F 2/966; A61F 2/82; A61F 2/95

(52) U.S. Cl. CPC A61F 2/966; A61F 2/82; A61F 2002/823; A61F 2002/9505

(57) ABSTRACT

An assembly for use in endoscopic stent delivery, including a stent delivery device and an associated stent loaded therein. The stent delivery device includes an outer tubular member, an inner member slidably disposed within the lumen of the outer tubular member, and a distal tip disposed at the distal end of the inner member. A self-expanding stent is positioned within the outer tubular member in a compressed configuration, with one or more suture loops constraining at least a portion of the stent. Upon deployment of the stent from the stent delivery device, proximal withdrawal of a release suture unravels the one or more suture loops.

### CROSS REFERENCE TO RELATED APPLICATIONS

This application claims the benefit of and priority to U.S. Provisional Patent Application Ser. No. 63/621,400, filed on Jan. 16, 2024, the disclosure of which is incorporated herein by reference.

### TECHNICAL FIELD

The disclosure pertains to medical devices and more particularly to stent delivery systems including a stent constraining structure configured to radially constrain a portion of a stent.

### BACKGROUND

A wide variety of medical devices have been developed for medical use including, for example, medical devices utilized to deliver stents. These medical devices may be used in a variety of locations and are manufactured and used according to any one of a variety of different methods. Accuracy of deployment of a stent has been found challenging, as many times the deployed stent is not properly positioned across the treatment site once deployed. Accordingly, there is an ongoing need to provide alternative stent delivery devices as well as alternative methods for manufacturing and using the stent delivery devices to control deployment of a stent across a treatment site.

### SUMMARY

This disclosure provides design, material, manufacturing method, and use alternatives for stent delivery systems.

One example is a stent delivery system. The system includes an outer tubular member having a proximal end, a distal end, and a lumen extending to the distal end, an inner member slidingly disposed within the lumen of the outer tubular member, and a distal tip disposed at a distal end of the inner member. The system also includes a self-expanding stent having a proximal end, a distal end, and a lumen extending therethrough. The self-expanding stent is expandable from a radially compressed configuration to a radially expanded configuration. A suture array is disposed around at least a portion of the self-expanding stent. In the radially compressed configuration, the self-expanding stent is disposed between an outer surface of the inner member and an inner surface of the outer tubular member. When the outer tubular member is withdrawn proximally, the self-expanding stent radially expands. When in the expanded configuration, at least a portion of the self-expanded stent is radially constrained by the suture array.

Alternatively or additionally to any of the examples herein, the suture array comprises one or more suture loops and a release knot connected to a release suture.

Alternatively or additionally to any of the examples herein, the release suture extends longitudinally through the lumen of the outer tubular member.

Alternatively or additionally to any of the examples herein, the release suture further comprises a release handle extending out from the proximal end of the outer tubular member.

Alternatively or additionally to any of the examples herein, the when the release handle is withdrawn in a proximal direction, the release knot is untied and the suture array unravels.

Alternatively or additionally to any of the examples herein, the release handle comprises a suture loop, configured to allow a user to grip the release handle.

Alternatively or additionally to any of the examples herein, the release knot is at a proximal end of the suture array.

Alternatively or additionally to any of the examples herein, the release knot is at a distal end of the suture array.

Alternatively or additionally to any of the examples herein, the suture array is disposed around a medial region of the self-expanding stent.

Alternatively or additionally to any of the examples herein, a proximal end region of the self-expanding stent is devoid of the suture array, and a distal end region of the self-expanding stent is devoid of the suture array.

Alternatively or additionally to any of the examples herein, when the outer tubular member is withdrawn proximally the proximal end region of the self-expanding stent radially expands to the expanded configuration and the distal end region of the self-expanding stent radially expands to the expanded configuration, while the medial region of the self-expanded stent remains radially constrained by the suture array.

Alternatively or additionally to any of the examples herein, the self-expanding stent further comprises a coating disposed around at least a portion of the stent.

Alternatively or additionally to any of the examples herein, when in the expanded configuration, at least a portion of the self-expanding stent is radially constrained by the coating.

Alternatively or additionally to any of the examples herein, the coating is dissolvable.

Another example is a stent delivery system. The system includes an outer tubular member having a proximal end, a distal end, and a lumen extending to the distal end, an inner member slidingly disposed within the lumen of the outer tubular member, and a distal tip disposed at a distal end of the inner member. The system also includes a self-expanding stent having a proximal end, a distal end, and a lumen extending therethrough. The self-expanding stent is expandable from a radially compressed configuration to a radially expanded configuration. A stent constraining member is disposed around a portion of the self-expanding stent. The outer tubular member is axially movable relative to the inner member between a delivery configuration and a deployment configuration. In the delivery configuration the outer tubular member surrounds the self-expanding stent such that the self-expanding stent is constrained between an outer surface of the inner member and an inner surface of the outer tubular member. In the deployment configuration the outer tubular member is withdrawn proximally of the self-expanding stent while the portion of the self-expanding stent remains constrained in the radially compressed configuration by the stent constraining member.

Alternatively or additionally to any of the examples herein, the stent constraining member comprises a suture array wrapped around the portion of the self-expanding stent.

Alternatively or additionally to any of the examples herein, the stent constraining member comprises a polymer covering surrounding the portion of the self-expanding stent, the polymer covering configured to delay expansion of the portion of the self-expanding stent.

Another example is a stent delivery system. The system includes an outer tubular member having a proximal end, a distal end, and a lumen extending to the distal end. An inner member is slidingly disposed within the lumen of the outer tubular member. A distal tip is disposed at a distal end of the inner member. A self-expanding stent is positionable around a distal region of the inner member proximal of the distal tip. The self-expanding stent has a proximal end, a distal end, and a lumen extending therethrough. The self-expanding stent is expandable from a radially compressed configuration to a radially expanded configuration. A suture array is disposed around a medial region of the self-expanding stent. The self-expanding stent is constrained in the radially compressed configuration when the self-expanding stent is disposed between an outer surface of the inner member and an inner surface of the outer tubular member. When the outer tubular member is withdrawn proximally, a proximal end region of the self-expanding stent radially expands and a distal end region of the self-expanding stent radially expands, while the medial region of the self-expanding stent remains constrained in the radially compressed configuration by the suture array.

Another example is a method of deploying a stent across a stricture within a body lumen. The method includes advancing a stent delivery catheter through the body lumen such that a self-expanding stent disposed within an outer tubular member of the stent delivery catheter crosses the stricture, and thereafter, withdrawing the outer tubular member proximally relative to the self-expanding stent such that a distal region of the self-expanding stent radially expands distal of the stricture and a proximal end region of the self-expanding stent radially expands proximal of the stricture while a medial region of the self-expanding stent remains radially constrained by a suture array. Thereafter, the suture array is released to allow the medial region of the self-expanding stent to radially expand across the stricture.

Alternatively or additionally to any of the examples herein, the method includes pulling a release suture proximally to untie a release knot in order to release the suture array.

Alternatively or additionally to any of the examples herein, the medial region of the self-expanding stent is positioned within the stricture prior to withdrawing the outer tubular member proximally relative to the self-expanding stent.

The above summary of some embodiments, aspects, and/or examples is not intended to describe each embodiment or every implementation of the present disclosure. The figures and the detailed description which follows more particularly exemplify these embodiments.

### BRIEF DESCRIPTION OF THE DRAWINGS

The disclosure may be more completely understood in consideration of the following detailed description of various embodiments in connection with the accompanying drawings, in which:

FIG. 1 illustrates a perspective view of an embodiment of a stent delivery system;

FIGS. 2A-2D illustrate a stent foreshortening when being deployed from a conventional stent delivery catheter;

FIG. 3 illustrates a stent delivery system including a stent constraining structure.

FIGS. 4A-4D illustrate aspects of deploying a stent with a stent delivery system having a stent constraining structure.

FIG. 5 illustrates another stent delivery system including stent constraining structures.

FIGS. 6A-6B illustrate aspects of a stent having a stent constraining structure when deployed in a body lumen.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “withdraw”, variants thereof, and the like, may be generally considered with respect to the positioning, direction, and/or operation of various elements relative to a user/operator/manipulator of the device, wherein “proximal” and “withdraw” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device.

The term “extent” may be understood to mean a greatest measurement of a stated or identified dimension, unless the extent or dimension in question is preceded by or identified as a “minimum”, which may be understood to mean a smallest measurement of the stated or identified dimension. For example, “outer extent” may be understood to mean a maximum outer dimension, “radial extent” may be understood to mean a maximum radial dimension, “longitudinal extent” may be understood to mean a maximum longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently-such as, but not limited to, angularly, radially, circumferentially (e.g., along an arc), etc. Additionally, the term “substantially” when used in reference to two dimensions being “substantially the same” shall generally refer to a difference of less than or equal to 5%.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The following description should be read with reference to the drawings, which are not necessarily to scale, wherein similar elements in different drawings are numbered the same. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

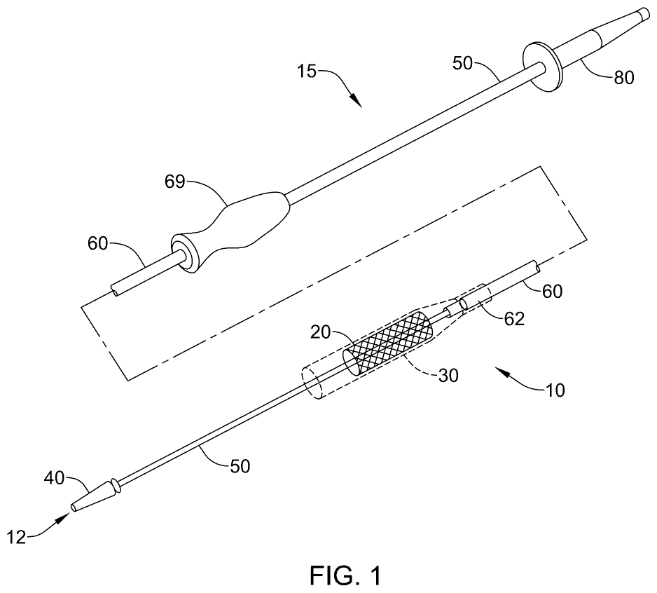

With reference to the drawings, FIG. 1 shows a perspective view of a stent delivery system 10 in accordance with an embodiment of the disclosure. The stent delivery system 10 may include a stent delivery device 15 having a stent 20 loaded therein for delivery to an anatomical site. As seen in FIG. 1, the stent 20 is loaded within an outer sheath 30 which may be attached to or be a portion of an outer tubular member 60 extending proximally to a distal handle 69. In some instances, the outer sheath 30 may be formed separately from the outer tubular member 60 and secured thereto at an attachment joint 62. In other instances, the outer sheath 30 may be a distal end region of the outer tubular member 60 sized to surround the compressed stent 20 prior to deployment. The stent delivery catheter 15 may include an inner member 50 slidably disposed within the lumen of the outer tubular member 60. A distal tip 40 may be secured to or otherwise incorporated with the distal end of the inner member 50. The distal tip 40 may have a distal end 12 at the distalmost extent of the distal tip 40 and the stent delivery device 15. The inner member 50 may extend through the outer tubular member 60 with a proximal end region of the inner member 50 secured to a proximal handle 80. The proximal handle 80 may be located proximal of the distal handle 69 such that the distal handle 69 may be axially translated relative to the proximal handle 80 to longitudinally actuate the outer sheath 60 relative to the stent 20 and the inner member 50, and thus deploy the stent 20. It is noted, that when the stent 20 is radially constrained around the inner member 50 and loaded in the outer sheath 30 for delivery, the distal end of the outer sheath 30 may abut and/or surround a proximal end region of the distal tip 40. While aspects of the present disclosure can be applied to the delivery of many intraluminary devices, it will be described for use in delivering a self-expanding stent 20.

A stent 20 is generally capable of being radially compressed and longitudinally extended when being delivered within the outer sheath 30 for implantation into a body lumen. The degree of elongation may depend upon the structure and intended function of the stent, and may be quite varied. The stent 20 may be constructed to self-expand when released from a radially compressed state (e.g., when a radially restraining force is removed from the stent by withdrawing the outer sheath 30 therefrom). Further, the stent 20 may be repositionable, removeable, and/or reconstrainable in some instances. In some instances, the stent 20 may include one or more, or a plurality of interwoven wires or filaments, forming a braided construction, a knitted construction, or other interwoven construction. In other instances, the stent 20 may be a monolithic structure including a plurality of interconnected struts and interstitial spaces therebetween. Thus, various stent types and constructions may be employed, and the stent delivery system 10 can be constructed to accommodate stents of various sizes and configurations.

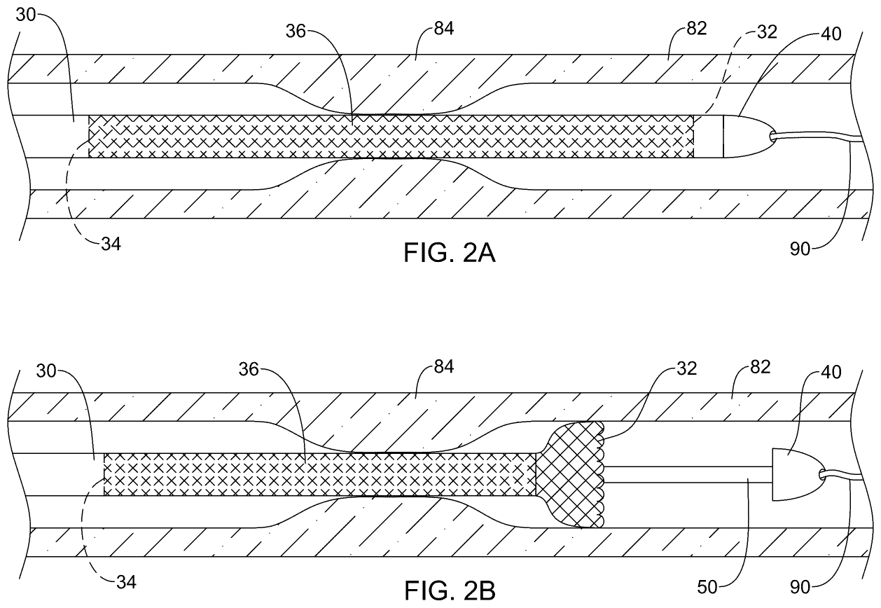

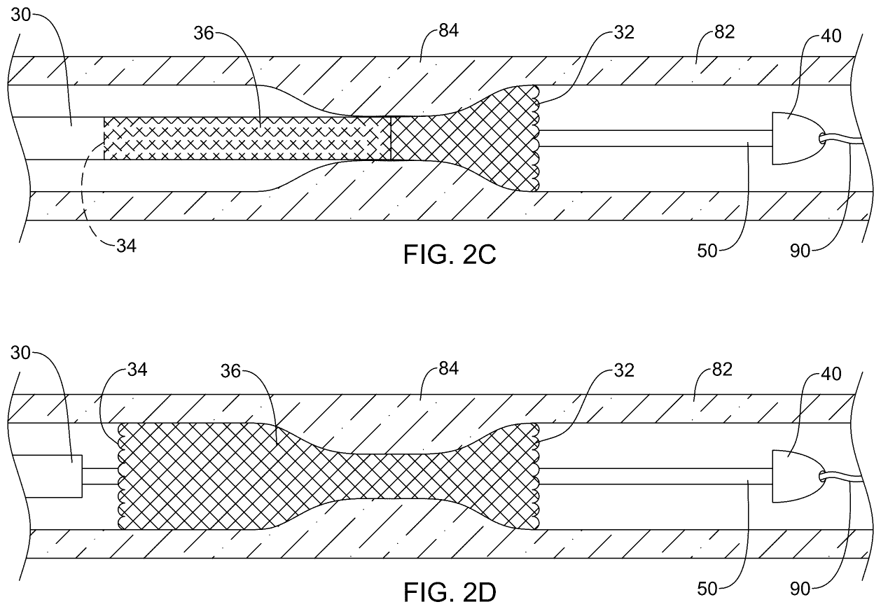

As illustrated in FIGS. 2A-2D, conventional stent delivery systems are prone to stent foreshortening during deployment of the stent. As shown in FIG. 2A, during delivery (e.g., delivery over a guidewire 90), the stent may be positioned in a radially constrained, longitudinally elongated configuration within the distal end region of the outer tubular member 60 (e.g., within the outer sheath 30) such that the radially constrained stent closely surrounds the inner member 50. In the radially constrained configuration, the distal end 32 of the stent is positioned adjacent the distal tip 40. The distal end of the outer sheath 30 may abut and/or surround a proximal end region of the distal tip 40. The stent delivery catheter may be advanced through a body lumen 82 such that the radially constrained stent 20 is positioned across a stricture 84. Thus, the distal end region 32 of the constrained stent 32 may be located distal of the stricture 84 and the proximal end region 34 of the constrained stent 32 may be located proximal of the stricture 84. Withdrawal of the outer tubular member 60 (and thus the outer sheath 30) in the proximal direction relative to the inner member 50, distal tip 40 and the stent moves the distal end of the outer sheath 30 proximal of the distal tip 40 to expose the distal end region 32 of the stent 20. As the outer sheath 30 is proximally withdrawn, the exposed distal end region 32 of the stent 20 is released from the radially constrained configuration, as shown in FIG. 2B. As the exposed distal end region 32 of the stent 20 begins to radially expand, the exposed distal end region 32 of the stent also begins to foreshorten in the proximal direction. In other words, the distal end region 32 of the stent 20, when deployed, moves proximally away from the distal tip 40. The central region 36 of the stent 20 also begins to move in the proximal direction, away from the distal tip 40. Further proximal withdrawal of the outer sheath 30, further deploys an additional length of the stent 20 to radially expand as the additional length of the stent 20 is exposed, as shown in FIG. 2C. As the exposed length of the stent 20 increases, the amount of foreshortening of the stent 20 likewise increases, moving the distal end region 32 of the stent 20 further proximally away from the distal tip 40 as the stent 20 radially expands and longitudinally foreshortens. Further foreshortening of the stent 20 as addition length of the stent 20 is exposed also continues to shift the central region 36 of the stent 20 proximally. As the central region 36 of the stent 20 moves proximally during deployment of the stent 20, it is difficult to accurately position the stent 20 across the stricture 84. As shown in FIG. 2D, when fully deployed, the stent is off-centered from the desired deployment location, (e.g. stricture). For example, the central region 36 may be deployed too proximal or too distal of the stricture 84 such that the stent 20 does not extend across the stricture 84 in a desired position. In some embodiments, the user may be able to compensate for stent foreshortening by advancing the stent delivery catheter such that the stent 20 is past the intended deployment location to account for the axial displacement of the distal end region of the stent 20 (i.e., foreshortening) during deployment. However, in many instances, accurate positioning of the expanded stent 20 across the stricture 84 may not be attained. Improper stent deployment or placement may cause complications, and/or require removal and replacement of the stent.

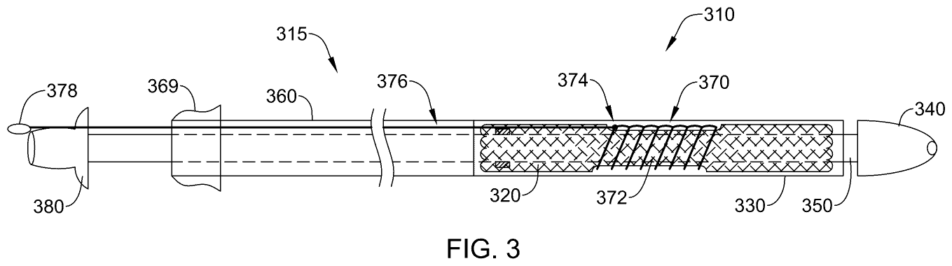

The foreshortening as shown in FIGS. 2A-2D can be remedied with the addition of a stent constraining member, such as a stent constraining suture, a band, a covering, or other structural element, disposed around a portion of the self-expanding stent 20. FIG. 3 illustrates a stent delivery system 310, which may be similar to the stent delivery system 10 in many aspects. For example, the stent delivery system 310 may include a stent delivery catheter 315 and stent 320 loaded therein for deployment at a treatment site. The stent delivery catheter 315 may include an outer tubular member 360 defining an outer sheath 330 at a distal end thereof. As noted above, the outer sheath 330, which may surround the stent 320, may be formed unitarily as a distal end region of the outer tubular member 360, may be a separate component attached to the outer tubular member 360, or may otherwise be provided at the distal end of the outer tubular member 360. The stent delivery catheter 315 may also include an inner member 350 slidably disposed in the lumen of the outer tubular member 360. A distal tip 340 may be disposed at the distal end of the inner member 350. The stent 320 may be loaded into the stent delivery catheter 315 with the stent 320 radially compressed around a distal end region of the inner member 350 proximal of the distal tip 340 such that the radially compressed stent 320 is positioned radially between an outer surface of the inner member 350 and an inner surface of the outer tubular member 360 in a delivery configuration.

The proximal end of the outer tubular member 360 may be secured to a first, distal handle 369 and the proximal end of the inner member 350 may be secured to a second, proximal handle 380. Longitudinal actuation of the first handle 369 relative to the second handle 380 may longitudinally translate the outer tubular member 360 relative to the inner member 350. For example, proximal actuation of the outer tubular member 360 relative to the inner member 350 may move the distal end of the outer tubular member 360 proximal of the stent 320 into a deployment configuration in which the stent 320 is exposed from the outer tubular member 360. In the deployment configuration in which the outer tubular member is withdrawn proximally of the self-expanding stent, a portion of the self-expanding stent constrained by the stent constraining member remains constrained in the radially compressed configuration by the stent constraining member

In one embodiment the stent constraining member may be a stent constraining suture, as shown in FIG. 3 and described herein as a suture array 370. The suture array 370 may include one or more sutures wrapped around a portion of the stent 320 and positioned within the outer sheath 330 and around the inner tubular member 350 when the stent 320 is loaded therein. The suture array 370 may surround any desired portion of the stent 320, and in some instances surrounds a first portion of the stent 320 while a remaining portion of the stent 320 is not radially constrained by the suture array 370. For example, as shown in FIG. 3, a medial region of the stent 320 may be radially constrained by the suture array 370 while a proximal end region and/or a distal end region of the stent 320 is devoid of the suture array 370 and thus not radially constrained by the suture array 370. In other instances, only the proximal end region may be radially constrained by the suture array 370, only the distal end region may be radially constrained by the suture array 370, or both the proximal and distal end regions may be radially constrained by the suture array 370 while a medial region of the stent 320 remains radially unconstrained by the suture array 370.

The suture array 370 may surround the stent 320 and be configured in any desired fashion to be releasable from the stent 320 to permit the portion of the stent 320 constrained by the suture array 370 to radially expand. For instance, the suture array 370 may be made up of any number of suture linkage loops 372 circumferentially surrounding the stent 320. The suture array 370 may include an actuation mechanism configured to be actuated by a user in order to release the suture array 370 from the stent 320 to permit the underlying portion of the stent 320 to radially expand. For example, the suture array 370 may include a pull member, such as a release suture 376 connected to a release knot 374. The release suture 376 and release knot 374 may be located at any position along the suture array 370, and there may be any number of release sutures 376 and/or knots 374 provided. For example, in some instances the release knot 374 may be provided at a proximal end of the suture array 370. In other instances, the release knot 374 may be provided at a distal end of the suture array 370, or at another desired location. Actuation of the pull member, such as the release suture 376, may untie, unwrap, or otherwise release the suture array 370 from radially constraining the stent 320. For instance, actuation of the pull member, such as the release suture 376, may untie or release the release knot 374 to thereby release the suture array 370 from radially constraining the stent 320. In some instances, a release handle 378 may be connected to a proximal end of the release suture 376, or other pull member, and be accessible by a user to actuate the release suture 376 or other pull member. The release handle 378 may be any manner of loop, knob, bar, lever or other design that gives a user the capability to release the suture array 370 when desired.

The release suture 376, or other pull member, may extend proximally from the suture array 370 wrapped around the constrained portion of the stent 320 to a proximal portion of the stent delivery catheter 315 for manipulation by a user. For example, the release suture 376, or other pull member, may extend through the lumen of the outer tubular member 360 to a location proximate the handle 369 or the handle 380. In other instances, the release suture 376, or other pull member, may extend through a lumen of the inner member 350 to a location proximate the handle 369 or the handle 380.

The suture array 370 may be configured to be released from the stent 320 independent of retracting the outer tubular member 360 proximally relative to the inner member 350 and stent 320. In other words, the stent 320 may be exposed from the outer tubular member 360 by proximally retracting the outer tubular member 360 to expose the stent 320 from a distal end thereof, while the portion of the stent 320 radially constrained by the suture array 370 remains constrained.

During delivery (e.g., delivery over a guidewire), the stent 320 may be positioned in a radially constrained, longitudinally elongated configuration within the distal end region of the outer tubular member 360 (e.g., within the outer sheath 330) such that the radially constrained stent 320 closely surrounds the inner member 350. The distal end of the outer sheath 330 may abut and/or surround a proximal end region of the distal tip 340. The suture array 370 may further constrain a portion, such as a central portion, of the stent 320. In some embodiments, the suture array 370 may be arranged toward one end of the stent 320, or there may be multiple arrays 370 in multiple locations along the length of the stent 320. As noted above, a suture array 370 may surround a proximal end region and/or a distal end region of the stent 320. The number and placement of suture arrays 370 may aid a physician in advancing stent placement distally post-deployment, or pulling the stent proximally to reposition the stent after it has been deployed. The one or more suture arrays 370 may have the same, or different release mechanisms, such as release sutures 376 and/or release handles 378 to allow for controlled release of the suture arrays 370 independently of one another and/or at different times.

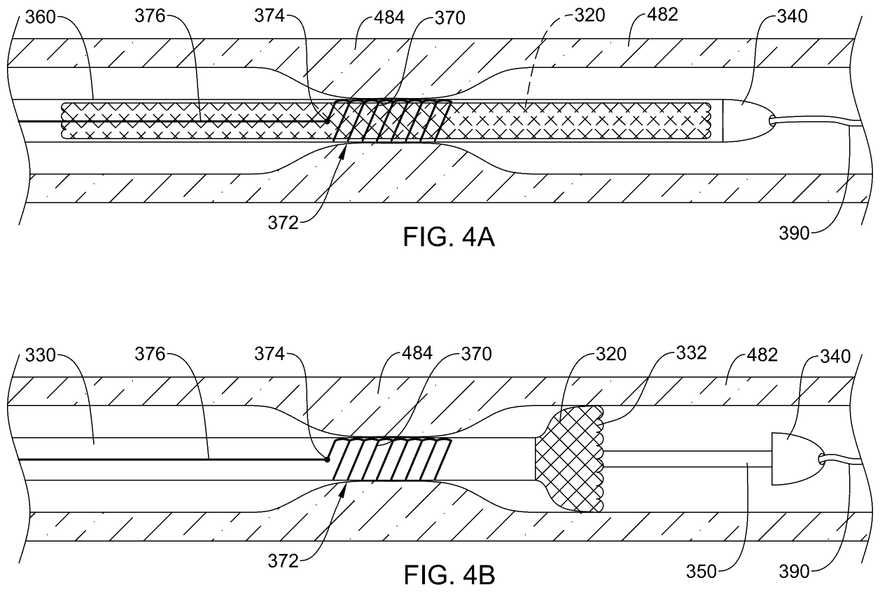

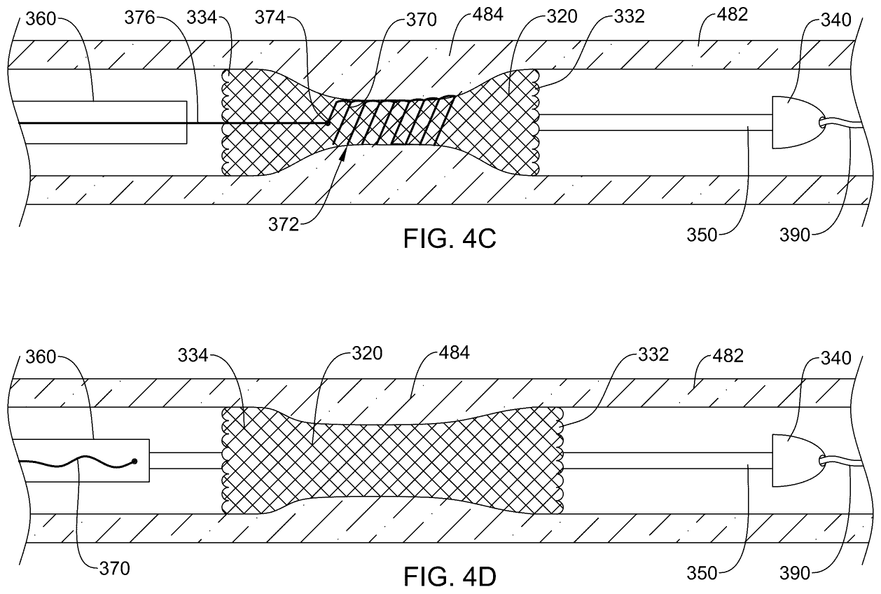

FIGS. 4A-4D illustrate aspects of delivering and deploying the stent 320 across a stricture 484 of a body lumen 482 using the stent delivery system 310. The stent delivery device 315 may be advanced through the body lumen 482 with the stent 320 in the delivery configuration with the stent 320 loaded in the outer tubular member 360 (e.g., the outer sheath 330 of the outer tubular member 360) prior to stent deployment. In some instances, the stent delivery system 310 may be advanced over a guidewire 390 extending through a guidewire lumen of the stent delivery device 315. As shown in FIG. 4A, the stent delivery device 315 may be positioned such that the stent constraining member (e.g., the suture array 370) is in a predetermined position relative to the stricture 484 (e.g. ensuring the center of the stent 320 is approximately centered across the stricture). In some instances the stent 320 and/or the stent delivery device 315 may include one or more radiopaque markers to visualize the placement of the stent 320 across the stricture 484. In addition or alternatively, the suture array 370 or other stent constraining structure may be formed of and/or seeded with radiopaque material to aid in stent placement and allow for visualization of the stent 320 during and after deployment.

Withdrawal of the outer tubular member (e.g., outer sheath 330) in the proximal direction relative to the inner member 350, distal tip 340 and the stent 320 moves the distal end of the outer sheath 330 proximal of the distal tip 340 to expose the distal end region of the stent 432, as shown in FIG. 4B. Distal foreshortening is observed, and while the overall length of the stent 320 decreases, the portion of the stent 320 (e.g. a medial region of the stent) radially constrained by the suture array 370 (or other stent constraining member) stays in place. In other words, the length of the constrained portion of the stent 320 surrounded by and radially constrained by the suture array 370 (or other stent constraining member) is not longitudinally displaced relative to the inner member 350, and thus is not longitudinally displaced relative to the stricture 484.

As the distal end of the outer tubular member (e.g., outer sheath 330) is further withdrawn proximally, more of the length of the stent 320 is exposed from the distal end of the outer sheath 330, until the entire length of the stent 320 is exposed, as shown in FIG. 4C. Portions of the stent 320 not surrounded by the suture array 370 (or other stent constraining member) are able to instantly and automatically radially expand once exposed from the distal end of the outer sheath 330, whereas the portion of the stent surrounded by and radially constrained by the suture array 370 (or other stent constraining member) remain constrained in the radially compressed configuration by the suture array 370 (or other stent constraining member).

After the stent 320 has been deployed out of the outer sheath 330 of the outer tubular member 360, the suture array 370 (or other stent constraining member) may be manipulated to subsequently permit the constrained portion of the stent 320 to radially expand. For example, when the entire length of the stent 320 has been exposed by the proximal retraction of the outer sheath 330, the suture array 370 may be untied, unraveled or otherwise released by actuating the release suture 376 (e.g., by proximal withdrawal of the suture release handle 378), which undoes (e.g., unties) the release knot 374 or otherwise releases the suture array 370 from radially constraining the portion of the sent the suture array 370 was surrounding, as shown in FIG. 4D. Upon untying, unraveling, or otherwise releasing the suture array 370 and withdrawing the suture array 370, the portions of the stent 320 that were constrained by the suture array 370 may radially expand further to enlarge the passage through the stricture 484. Prior to deployment, a physician may decide which portion of the stent 320 they want to remain constrained after deployment (if any). The portion of the stent 320 that they wish to keep constrained may differ from procedure to procedure as the anatomical dimensions differ in each person and type of procedure.

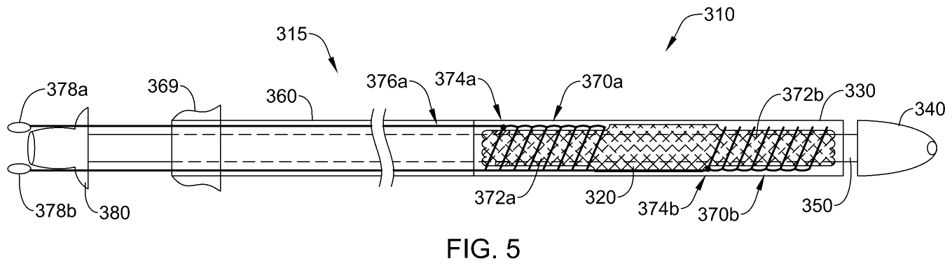

FIG. 5 illustrates the stent delivery system 310 including another configuration of stent constraining structures for constraining portions of the stent 320. The stent delivery system 310 may be similar to the stent delivery system 310 illustrated in FIG. 3 in many aspects. As shown in FIG. 5, multiple stent constraining structures may be disposed at multiple spaced apart locations along the length of the stent 320. For example, a first stent constraining member, shown as a first suture array 370a, may surround and radially constrain a proximal end region of the stent 320 and/or a second stent constraining member, shown as a second suture array 370b, may surround and radially constrain a distal end region of the stent 320. A medial region of the stent 320, located between the first suture array 370a (or other stent constraining member) and the second suture array 370b (or other stent constraining member) may be devoid or free of a stent constraining member, and thus not radially constrained by the suture arrays 370a, 370b.

Each of the suture arrays 370a, 370b may be formed and function similar to the suture array 370 discussed above. For example, the suture arrays 370a, 370b may surround the stent 320 and be configured in any desired fashion to be releasable from the stent 320 to permit the portion of the stent 320 constrained by the suture array 370a, 370b to radially expand. For instance, each of the suture arrays 370a, 370b may be made up of any number of suture linkage loops 372a, 372b circumferentially surrounding the stent 320. Each suture array 370a, 370b may include an actuation mechanism configured to be actuated by a user in order to release the suture array 370a, 370b from the stent 320 to permit the underlying portion of the stent 320 to radially expand. For example, the suture arrays 370a, 370b may include a pull member, such as a release suture 376a, 376b connected to a release knot 374a, 374b. The release suture 376a, 376b and release knot 374a, 374b may be located at any position along the suture array 370a, 370b, and there may be any number of release sutures 376a, 376b and/or knots 374a, 374b provided. Actuation of the pull member, such as the release suture 376a, 376b, may untie, unwrap, or otherwise release the corresponding suture array 370a, 370b from radially constraining the stent 320. For instance, actuation of the pull member, such as the release suture 376a, may untie or release the release knot 374a to thereby release the first suture array 370a from radially constraining the proximal end region of the stent 320 and/or actuation of the pull member, such as the release suture 376b, may untie or release the release knot 374b to thereby release the second suture array 370b from radially constraining the distal end region of the stent 320.

In some instances, a release handle 378a, 378b may be connected to a proximal end of the respective release suture 376a, 376b, or other pull member, and be accessible by a user to actuate the release suture 376a, 376b or other pull member. The release handle 378a, 378b may be any manner of loop, knob, bar, lever or other design that gives a user the capability to release the suture array 370a, 370b when desired.

The release suture 376a, 376b, or other pull member, may extend proximally from the respective suture array 370a, 370b wrapped around the constrained portion of the stent 320 to a proximal portion of the stent delivery catheter 315 for manipulation by a user. For example, the release suture 376a, 376b, or other pull member, may extend through the lumen of the outer tubular member 360 to a location proximate the handle 369 or the handle 380. In other instances, the release suture 376a, 376b, or other pull member, may extend through a lumen of the inner member 350 to a location proximate the handle 369 or the handle 380.

In some instances, the first suture array 370a may be configured to be untied, unwound, or otherwise released from the stent 320 independent of the second suture array 370b. In other instances, a single pull member, such as a single release suture, may be actuated to simultaneously or sequentially release both the first suture array 370a and the second suture array 370b.

Each of the suture arrays 370a, 370b may be configured to be released from the stent 320 independent of retracting the outer tubular member 360 proximally relative to the inner member 350 and stent 320. In other words, the stent 320 may be exposed from the outer tubular member 360 by proximally retracting the outer tubular member 360 to expose the stent 320 from a distal end thereof, while the portions of the stent 320 radially constrained by the suture arrays 370a, 370b remain constrained. Thereafter, the suture arrays 370a, 370b may be released from the respective constrained portions of the stent 320 to permit those underlying portions of the stent 320 to radially expand.

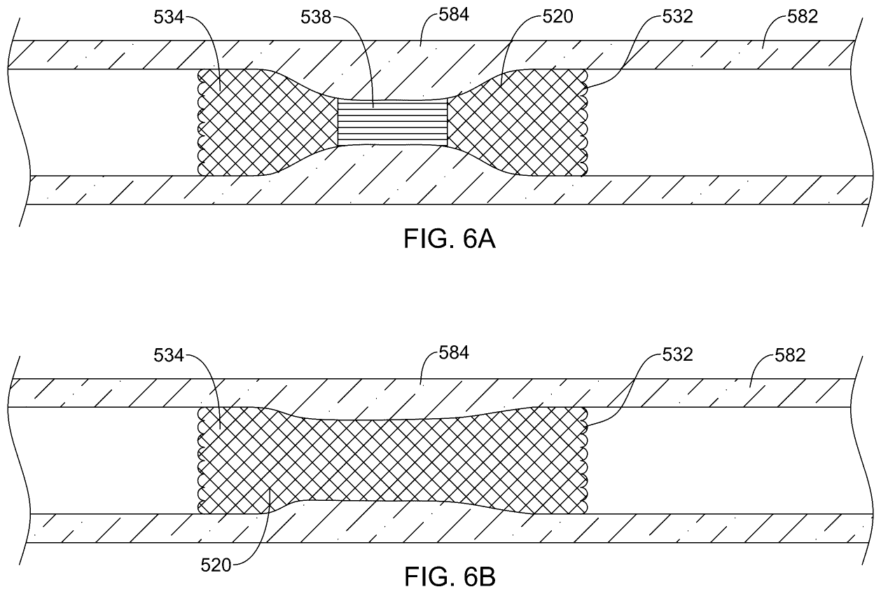

Other stent constraining members may be utilized to selectively constrain a portion of a stent upon deployment of the stent across a stricture. For example, as shown in FIG. 6A, a portion of a stent 520 may be constrained by a covering (e.g. coating) 538 which may temporarily hold a portion of the stent 520 in its constrained configuration after deployment from a stent delivery catheter (e.g. after being deployed from the distal end of an outer tubular member of a stent delivery catheter as discussed above). As shown in FIG. 6A, in some instances, the stent 520 may be deployed across a stricture 584 of a body lumen 582 such that the constrained portion of the stent 520 constrained by the covering 538 extends across the stricture 584. Although FIG. 6A illustrates a medial region of the stent 520 may be radially constrained by the covering 538 while a proximal end region and/or a distal end region of the stent 520 is devoid of the covering 538 and thus not radially constrained by the covering 538, in other instances, only the proximal end region may be radially constrained by the covering 538, only the distal end region may be radially constrained by the covering 538, or both the proximal and distal end regions may be radially constrained by the covering 538 while a medial region of the stent 520 remains radially unconstrained by the covering 538.

The covering (e.g. coating) 538 may be configured to dissolve, degrade, or be absorbed over time, or the covering (e.g., coating) 538 may remain intact to keep a portion of the stent 538 radially constrained indefinitely. As shown in FIG. 6B, at a later time, post-deployment of the stent 520 in the body lumen 582, the covering 538 may be released (e.g., absorbed or dissolved) from the stent 520, permitting the underlying portion of the stent 520 to radially expand. In some instances the covering, or portions thereof, may be formed of a dissolvable material, such as gelatin, glucose or similar water-soluble substances that dissolves over time to release the constrained portion of the stent 520. The applied covering 538 may also be seeded with or otherwise include radiopaque materials to aid in correct stent placement across the stricture 584 in the body lumen 582. The covering 538 may be used alone or in combination with a suture array 370 as discussed above.

It will be understood that the dimensions described in association with the above figure are illustrative only, and that other dimensions of slits and filter sheaths are contemplated. The materials that can be used for the various components of the stent delivery device for capturing lesion particles (and/or other systems or components disclosed herein) and the various elements thereof disclosed herein may include those commonly associated with medical devices. For simplicity purposes, the following discussion makes reference to the stent delivery device (and variations, systems or components disclosed herein). However, this is not intended to limit the devices and methods described herein, as the discussion may be applied to other elements, members, components, or devices disclosed herein.

In some embodiments, the stent delivery device (and variations, systems or components thereof disclosed herein) may be made from a metal, metal alloy, ceramics, zirconia, polymer (some examples of which are disclosed below), a metal-polymer composite, combinations thereof, and the like, or other suitable material. Some examples of suitable metals and metal alloys include stainless steel, such as 444V, 444L, and 314LV stainless steel; mild steel; nickel-titanium alloy such as linear-elastic and/or super-elastic nitinol; cobalt chromium alloys, titanium and its alloys, alumina, metals with diamond-like coatings (DLC) or titanium nitride coatings, other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R44035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R44003 such as ELGILOY®, PHYNOX®, and the like); platinum enriched stainless steel; titanium; platinum; palladium; gold; combinations thereof; and the like; or any other suitable material.

In at least some embodiments, portions or all of the stent delivery device (and variations, systems or components thereof disclosed herein) may also be doped with, made of, or otherwise include a radiopaque material. Radiopaque materials are understood to be materials capable of producing a relatively bright image on a fluoroscopy screen or another imaging technique during a medical procedure. This relatively bright image aids a user in determining the location of the stent delivery device (and variations, systems or components thereof disclosed herein). Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the stent delivery device (and variations, systems or components thereof disclosed herein) to achieve the same result.

In some embodiments, the stent delivery device (and variations, systems or components thereof disclosed herein) and/or portions thereof, may be made from or include a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex® high-density polyethylene, Marlex® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, polyurethane silicone copolymers (for example, Elast-Eon® from AorTech Biomaterials or ChronoSil® from AdvanSource Biomaterials), biocompatible polymers, other suitable materials, or mixtures, combinations, copolymers thereof, polymer/metal composites, and the like. In some embodiments, the sheath can be blended with a liquid crystal polymer (LCP). For example, the mixture can contain up to about 6 percent LCP.

In some embodiments, the stent delivery device (and variations, systems or components thereof disclosed herein) may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vascoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A stent delivery system, the system comprising:
an outer tubular member having a proximal end, a distal end, and a lumen extending to the distal end;
an inner member slidingly disposed within the lumen of the outer tubular member;
a distal tip disposed at a distal end of the inner member;
a self-expanding stent having a proximal end, a distal end, and a lumen extending therethrough, the self-expanding stent being expandable from a radially compressed configuration to a radially expanded configuration;
a suture array disposed around at least a portion of the self-expanding stent;
wherein in the radially compressed configuration, the self-expanding stent is disposed between an outer surface of the inner member and an inner surface of the outer tubular member;
wherein when the outer tubular member is withdrawn proximally, the self-expanding stent radially expands; and
wherein when in the expanded configuration, at least a portion of the self-expanded stent is radially constrained by the suture array.

2. The system of claim 1, wherein the suture array comprises one or more suture loops and a release knot connected to a release suture.

3. The system of claim 2, wherein the release suture extends longitudinally through the lumen of the outer tubular member.

4. The system of claim 3, wherein the release suture further comprises a release handle extending out from the proximal end of the outer tubular member.

5. The system of claim 4, wherein the when the release handle is withdrawn in a proximal direction, the release knot is untied and the suture array unravels.

6. The system of claim 4, wherein the release handle comprises a suture loop, configured to allow a user to grip the release handle.

7. The system of claim 2, wherein the release knot is at a proximal end of the suture array.

8. The system of claim 2, wherein the release knot is at a distal end of the suture array.

9. The system of claim 1, wherein the suture array is disposed around a medial region of the self-expanding stent.

10. The system of claim 9, wherein a proximal end region of the self-expanding stent is devoid of the suture array, and a distal end region of the self-expanding stent is devoid of the suture array.

11. The system of claim 10, wherein when the outer tubular member is withdrawn proximally the proximal end region of the self-expanding stent radially expands to the expanded configuration and the distal end region of the self-expanding stent radially expands to the expanded configuration, while the medial region of the self-expanded stent remains radially constrained by the suture array.

12. The system of claim 1, wherein the self-expanding stent further comprises a coating disposed around at least a portion of the stent.

13. The system of claim 12, wherein when in the expanded configuration, at least a portion of the self-expanding stent is radially constrained by the coating.

14. The system of claim 12, wherein the coating is dissolvable.

15. A stent delivery system, the system comprising:
an outer tubular member having a proximal end, a distal end, and a lumen extending to the distal end;
an inner member slidingly disposed within the lumen of the outer tubular member;
a distal tip disposed at a distal end of the inner member;
a self-expanding stent having a proximal end, a distal end, and a lumen extending therethrough, the self-expanding stent being expandable from a radially compressed configuration to a radially expanded configuration;
a stent constraining member disposed around a portion of the self-expanding stent;
wherein the outer tubular member is axially movable relative to the inner member between a delivery configuration and a deployment configuration;
wherein in the delivery configuration the outer tubular member surrounds the self-expanding stent such that the self-expanding stent is constrained between an outer surface of the inner member and an inner surface of the outer tubular member; and
wherein in the deployment configuration the outer tubular member is withdrawn proximally of the self-expanding stent while the portion of the self-expanding stent remains constrained in the radially compressed configuration by the stent constraining member.

16. The system of claim 15, wherein the stent constraining member comprises a suture array wrapped around the portion of the self-expanding stent.

17. The system of claim 15, wherein the stent constraining member comprises a polymer covering surrounding the portion of the self-expanding stent, the polymer covering configured to delay expansion of the portion of the self-expanding stent.

18. A method of deploying a stent across a stricture within a body lumen, comprising:
advancing a stent delivery catheter through the body lumen such that a self-expanding stent disposed within an outer tubular member of the stent delivery catheter crosses the stricture;
withdrawing the outer tubular member proximally relative to the self-expanding stent such that a distal region of the self-expanding stent radially expands distal of the stricture and a proximal end region of the self-expanding stent radially expands proximal of the stricture while a medial region of the self-expanding stent remains radially constrained by a suture array;
thereafter, releasing the suture array to allow the medial region of the self-expanding stent to radially expand across the stricture.

19. The method of claim 18, further comprising: pulling a release suture proximally to untie a release knot in order to release the suture array.

20. The method of claim 18, wherein the medial region of the self-expanding stent is positioned within the stricture prior to withdrawing the outer tubular member proximally relative to the self-expanding stent.
